(19) United States
(12) Patent Application Publication
(10) Pub. No.: US 2003/0082669 A1
(43) Pub. Date: May 1, 2003

(54) Glyoxylate cycle enzymes as targets for antifungal drug development

(75) Inventors: Michael C. Lorenz, Roslindale, MA (US); Gerald R. Fink, Chestnut Hill, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Whitehead Institute for Biomedical Research, Cambridge, MA

(21) Appl. No.: 10/071,894

(22) Filed: Feb. 8, 2002

Related U.S. Application Data
(60) Provisional application No. 60/267,622, filed on Feb. 9, 2001.

(51) Int. Cl. C12Q 1/18

(52) U.S. Cl. 435/32

(57) ABSTRACT

The present invention relates to methods of identifying a drug that inhibits the virulence of a fungus comprising contacting the fungus with a drug to be assessed and determining whether an enzyme involved in the glyoxylate pathway of the fungus is inhibited in the presence of the drug, wherein if an enzyme of the glyoxylate pathway of the fungus is inhibited in the presence of the drug, then the drug inhibits or virulence of the fungus. In particular embodiments, the present invention relates to methods of identifying a drug that inhibits the virulence of a yeast; methods of identifying a drug that inhibits the virulence of C. albicans; and methods of treating an individual who is susceptible to fungal infection comprising administering to the individual a drug identified by the methods described herein.

### RELATED APPLICATIONS

This application claims the benefit of U.S. Provisional Application No. 60/267,622, filed on Feb. 9, 2001. The entire teachings of the above application are incorporated herein by reference.

### GOVERNMENT SUPPORT

The invention was supported, in whole or in part, by grant 5 RO1 GM40266 from the National Institutes of Health. The Government has certain rights in the invention.

### BACKGROUND OF THE INVENTION

The common human fungal commensal, C. albicans, is a benign inhabitant of the gastrointestinal tract that can be pathogenic when it grows in other parts of the body. Both in vivo and in vitro, C. albicans is phagocytosed by cells of the innate immune system, including neutrophils and macrophages. Neutropenic patients deficient in these cells, such as those undergoing immunosuppressive or chemotherapeutic regimes or those with leukemia or lymphoma, are particularly susceptible to systemic candidiasis (Wright, W. L. & Wenzel, R. P., Infect. Dis. Clin. North. Am., 11:411-425 (1997); Bodey, G. et al. Eur. J Clin. Microbiol. Infect. Dis., 11:99-109 (1992)), whereas T-cell deficient patients, such as those with AIDS, are primarily at risk for mucosal infections. Contact between C. albicans and phagocytic cells in vitro results in secretion of cytokines from the phagocytes and induction of hyphal growth in the fungal cells (Ashman, R. B. & Papadimitriou, J. M., Microbiol. Rev., 59:646-672 (1995); Lo, H. J. et al., Cell, 90:939-949 (1997)). Nevertheless, the regulation and extent of these changes is poorly understood on a molecular level.

A better understanding of pathogenic organisms such as fungus would make possible identification of drugs to treat such infections.

### SUMMARY OF THE INVENTION

Macrophages represent a primary defense against fungal infections. In vitro, macrophages readily ingest both Saccharomyces cerevisiae and Candida albicans. Described herein is a coculture system in which only yeast cells that have been phagocytosed have been isolated. RNA from such cells was used to probe Affymetrix DNA microarrays to assess expression changes after phagocytosis. The most highly induced genes were those encoding enzymes and associated factors for the glyoxylate cycle. This metabolic pathway, an offshoot of the tricarboxylic acid cycle, allows fungi to utilize two carbon compounds such as acetate or ethanol as the sole carbon source. Two enzymatic steps are specific to the glyoxylate cycle, isocitrate (ICL1) and malate synthase (MLS1), and the genes encoding these enzymes were identified in C. albicans. Northern analysis demonstrated that the C. albicans genes were also induced upon phagocytosis. Work described herein demonstrate that C. albicans strains lacking ICL1 or MLS1 are unable to utilize acetate or ethanol as carbon sources and have a filamentation defect in response to alkaline pH.

Accordingly, the present invention relates to methods of identifying a drug that inhibits an enzyme involved in the glyoxylate pathway of a microorganism, such as fungus, bacterium, mycobacterium (e.g., M. tuberculosis), parasite (e.g., parasitic worm, such as C. elegans), and further methods of identifying a drug that inhibits (reduces) the virulence of such microorganisms. The method comprises contacting the microorganism (e.g., fungus, bacterium, mycobacterium, parasite) with a drug to be assessed and determining the ability of the microorganism to utilize two-carbon compounds as the sole carbon source. If the ability of the microorganism to utilize two-carbon compounds as the sole carbon source is inhibited in the presence of the drug to be assessed, the drug is a drug that inhibits an enzyme in the glyoxylate cycle. In the method of identifying or aiding the identification of a drug that inhibits the virulence of a microorganism, a drug that inhibits the ability of the microorganism to utilize two-carbon compounds as the sole carbon source is further assessed for its ability to inhibit the virulence of the microorganism in an appropriate animal model, such as a murine model of systemic candidiasis.

In one embodiment, the present invention relates to methods of identifying a drug that inhibits (reduces) the virulence of a fungus comprising contacting the fungus (e.g., filamentous fungus, yeast) with a drug to be assessed and determining whether an enzyme involved in the glyoxylate pathway of the fungus is inhibited in the presence of the drug. If an enzyme of the glyoxylate pathway of the fungus is inhibited in the presence of the drug (e.g., itaconic acid), then the drug inhibits virulence of the fungus.

In one embodiment, the invention relates to a method of identifying a drug that inhibits the virulence of a yeast comprising contacting the yeast (e.g., S. cerevisiae, C. albicans) with a drug to be assessed and determining whether an enzyme involved in the glyoxylate pathway of the yeast is inhibited in the presence of the drug. If an enzyme of the glyoxylate pathway of the yeast is inhibited in the presence of the drug, then the drug inhibits virulence of the yeast.

In another embodiment, the invention relates to a method of identifying a drug that inhibits the virulence of C. albicans comprising contacting the C. albicans with a drug to be assessed and determining whether an enzyme involved in the glyoxylate pathway of the C. albicans is inhibited in the presence of the drug. If an enzyme of the glyoxylate pathway of the C. albicans is inhibited in the presence of the drug, then the drug inhibits virulence of the C. albicans.

In a particular embodiment, the present invention relates to a method of identifying a drug that inhibits the virulence of C. albicans comprising contacting the C. albicans with a drug to be assessed and determining whether isocitrate lyase of the C. albicans is inhibited in the presence of the drug. If the isocitrate lyase of the C. albicans is inhibited in the presence of the drug, then the drug inhibits virulence of the C. albicans. A drug that reduces the virulence of C. albicans can also be identified by contacting the C. albicans with a drug to be assessed and determining whether malate synthase of the C. albicans is inhibited in the presence of the drug. If the malate synthase of the C. albicans is inhibited in the presence of the drug, then the drug inhibits virulence of the C. albicans.

The drugs identified in the methods described herein are also encompassed by the present invention. The drugs identified in the methods of the present invention can be used to treat fungal infections. In one embodiment, the present invention relates to methods of treating an individual who is susceptible to fungal infection comprising administering to the individual a drug identified by the methods described herein. For example, an individual who is undergoing immunosuppressive or chemotherapeutic treatment can be treated with a drug identified by the methods described herein. In addition, an individual who is neutropenic or T-cell deficient (e.g., an individual infected with HIV) can be treated with a drug identified by the methods described herein. In a particular embodiment, the present invention relates to a method of treating a fungus infection in an individual comprising administering to the individual a therapeutically effective amount of a drug that inhibits an enzyme involved in the glyoxylate cycle of the fungus.

As a result of the work described herein, methods of identifying drugs for treating fungal infections as well as methods of treating fungal infections (e.g., thrush, vaginitis; individuals with leukemia, lymphoma, trauma, or undergoing chemotherapy/radiation, immunosurpressed individuals, individuals with medical implants such as heart valves, catheters) are provided.

### BRIEF DESCRIPTION OF THE DRAWINGS

FIGS. 1A-1B are the Northern analysis which shows that macrophage contact induces isocitrate lyase and malate synthase in both S. cerevisiae and C. albicans. RNA was prepared from yeast cells exposed to macrophages for three hours and hybridized to radiolabelled probes made from S. cerevisiae or C. albicans ICL1 (FIG. 1A) or MLS1 (FIG. 1B).

FIG. 2A shows the phenotypes of isocitrate lyase mutant strains. C. albicans strains SC5314 (wild-type), MLC6 (icl1/+), MLC7 (icl1/), and MLC10 (icl1/+ICL1), or S. cerevisiae strains EM93 (wild-type) and MLY283 (icl1/MATa/a) were incubated on YNB media containing 2% glucose (left) or 2% sodium acetate (right) as the sole carbon source. The glucose-grown cells were incubated at 37° C. for 2 days; acetate-grown cells for 4 days.

FIG. 2B are graphs showing C. albicans strains SC5314, MLC6, MLC7, and MLC10 that were grown in liquid YPD (left) or YNB containing 2% sodium acetate as the sole carbon source (right) at 37° C. for the indicated time.

FIGS. 3A-3C show that isocitrate lyase mutants are not stress sensitive. FIG. 3A shows C. albicans strains SC5314 (wild-type), MLC7 (icl1/) and MLC10(icl1/+ICL1) which were grown on YPD medium containing 1.5 M NaCl, 250 mM LiCl, 10% ethanol (at 37° C.) or on YPD at 42° C. for 30 hours. FIG. 3B shows liquid cultures of SC5314, MLC6 (icl1/+), MLC7 (icl1/) and MLC 10 (icl1/+ICL1) which were grown in YPD at 37° C. to OD600˜1.0, serially diluted (1:10 dilutions), and plated by spotting to media with YPD+5 mM H₂O₂ at 37° C. for 30 hours. FIG. 3C shows SC5314, MLC7, and MLC10 which were incubated on 2% agar/10% serum medium for 30 hours at 37° C. and photographed at 100× magnification.

FIG. 4 is a graph showing that isocitrate lyase mutants are avirulent. 5×106 cells of the indicated strains were injected into the tail vein of BALB/c mice (n=10) and monitored for 28 days.

FIG. 5 is an alignment of the amino acids sequences of isocitrate lyase from C. albicans ICL1, S. cerevisiae ICL1, C. tropicalis ICL, A. nidulans acuD, A. thaliana AceA and E. coli aceA (SEQ ID Nos: 1-6, respectively).

FIG. 6 shows that regulation of ICL1 is similar in both S. cerevisiae and C. albicans.

FIG. 7 shows that glyoxylate mutants cannot grow on acetate or ethanol.

FIG. 8 are graphs of the growth rates of C. albicans mutants.

FIG. 9 shows that itaconic acid inhibits growth on acetate.

### DETAILED DESCRIPTION OF THE INVENTION

The incidence of systemic fungal infections, which have a high mortality rate, has risen with the population of immunosuppressed patients. Candida albicans,a diploid asexual yeast and normal component of the mammalian gastrointestinal flora, is responsible for the majority of these infections. The mortality rate from these infections is high, due both to the severity of the underlying host condition and the poor efficacy of current therapies. The development of systemic disease must result from a failure of the weakened immune system to confine these organisms to their natural body sites. Described herein is an in vitro system in which live cells of the related yeast Saccharomyces cerevisiae are isolated from the phagolysosome of cultured mammalian macrophages. Analysis of global gene expression profiles in this population showed that the predominant response is the induction of the glyoxylate cycle, a metabolic pathway enabling microorganisms to utilize two-carbon compounds as carbon sources. In C. albicans, isocitrate lyase (ICL1) and malate synthase (MLS1), the principal enzymes of the glyoxylate cycle, are also upregulated upon phagocytosis. C. albicans (icl1/icl1) strains lacking ICL1 are markedly less virulent than the wild-type in a model of systemic candidiasis. These findings, in conjunction with recent reports that isocitrate is both upregulated in vivo and required for virulence in Mycobacterium tuberculosis (McKinney, J. D., et al. Nature, 406:735-738 (2000); Honer Zu Bentrup, et al., J. Bacteriol., 181:7161-7167 (1999)) demonstrate the wide-ranging significance of the glyoxylate cycle in microbial pathogenesis.

Accordingly, the present invention relates to methods of identifying a drug that inhibits an enzyme involved in the glyoxylate pathway of a microorganism, such as fungus, bacterium, mycobacterium (e.g., M. tuberculosis), parasite (e.g., parasitic worm, such as C. elegans), and further methods of identifying a drug that inhibits (reduces) the virulence of such microorganisms. The method comprises contacting the microorganism (e.g., fungus, bacterium, mycobacterium, parasite) with a drug to be assessed and determining the ability of the microorganism to utilize two-carbon compounds as the sole carbon source. If the ability of the microorganism to utilize two-carbon compounds as the sole carbon source is inhibited in the presence of the drug to be assessed, the drug is a drug that inhibits an enzyme in the glyoxylate cycle. In the method of identifying or aiding the identification of a drug that inhibits the virulence of a microorganism, a drug that inhibits the ability of the microorganism to utilize two-carbon compounds as the sole carbon source is further assessed for its ability to inhibit the virulence of the microorganism in an appropriate animal model, such as a murine model of systemic candidiasis.

The present invention also relates to methods of identifying a drug that inhibits the virulence of a fungus comprising contacting the fungus with a drug to be assessed and determining whether an enzyme involved in the glyoxylate pathway of the fungus is inhibited in the presence of the drug, wherein if an enzyme of the glyoxylate pathway of the fungus is inhibited in the presence of the drug, then the drug inhibits or virulence of the fungus. In particular embodiments, the present invention relates to methods of identifying a drug that inhibits the virulence of a yeast; methods of identifying a drug that inhibits the virulence of C. albicans; drugs identified by the methods of the present invention; and methods of treating an individual who is susceptible to fungal infection comprising administering to the individual a drug identified by the methods described herein.

The fungus can be any variety of fungi and can be a pathogenic fungus or a nonpathogenic fungus that can have an adverse effect in an individual. For example, the fungus can be normally nonpathogenic, but possess the ability to become virulent or virulent-like in an immunocompromised individual. The fungus can be a filamentous fungus species, including but not limited to Acremonium species, Aspergillus species, Claviceps species, Collertortichum species, Fusarium species, Monascue species, Neurospora species, Nodulisporium species, Penicillium species, Pestalotiopsis species, Taxomyces species, Tolypocladium species and Trichoderma species. In a particular embodiment, the fungus is a yeast such as S. cerevisiae, C. albicans, C. glabrata and C. krusei.

The phrase “inhibits the virulence of a fungus” includes partial or complete inhibition of the virulence of a fungus. For example, the virulence of the fungus can be reduced in the presence of the drug being assessed compared to the virulence of the same fungus which has not been exposed to the drug. Alternatively, the virulence of the fungus can be abolished in the presence of the drug.

A drug identified in the methods of the present invention can inhibit an enzyme involved in the glyoxylate pathway by inhibiting (partially or completely) the expression of a gene involved in the glyoxylate pathway. The drug can inhibit expression of the gene directly or indirectly. For example, the drug can directly inhibit expression of the gene by inhibiting transcription and/or translation of the gene, or indirectly inhibit expression of the gene by inhibiting another gene or protein (e.g., cofactor) required for expression of the gene involved in the glyoxylate pathway. In addition, a drug identified in the methods of the present invention can inhibit an enzyme involved in the glyoxylate pathway by inhibiting (partially or completely) the protein expressed by the gene involved in the pathway directly or indirectly. For example, the drug can directly inhibit the protein by binding to the protein (e.g., antibody) or indirectly inhibit the protein by inhibiting an upstream or downstream product required for function of the protein.

Examples of enzymes of the glyoxylate pathway that can be inhibited by a drug to be assessed include, but are not limited to, isocitrate lyase (ICL1), malate synthase synthase (MLS1), malate dehydrogenase (MDH2), citrate synthase (CIT2), acetyl-CoA synthase (ACS1), CRC1, ACR1, YAT1, YER024w, YDR384c and fructose-1,6-biphosphatase (FBP1).

Whether an enzyme involved in the glyoxylate pathway of a fungus is inhibited can be determined using a variety of methods. For example, an in vivo infectivity assay, such as the murine virulence assay described in the exemplification can be used.

Drugs which reduce virulence of a fungus can be used in methods of treatment. For example, drugs identified herein can be used to treat an individual who is susceptible to fungal infection comprising administering to the individual a drug identified by the methods described herein. For example, an individual who is undergoing immunosuppressive or chemotherapeutic treatment can be treated with a drug identified by the methods described herein. In addition, an individual who is neutropenic or T-cell deficient (e.g., an individual infected with HIV) can be treated with a drug identified by the methods described herein.

Experimentation

Methods

Yeast-Macrophage Coculture and Gene Expression Analysis

Murine macrophage-like cell line J774A (ATCC stock number TIB-67) was cultured in RPMI+10% fetal bovine serum at 37° C. in 95% air/5% CO2. ˜18 hour prior to a coculture experiment, cells were plated in 50 ml media at 2×10⁷ cells/750 ml flask. Yeast strain EM93 (MATa/α prototroph (Mortimer, R. K. & Johnston, J. R., Genetics, 113:35-43 (1986)), was grown overnight in YPD at 37° C., then diluted in fresh YPD for 3-4 hours (˜2 doublings). Yeast cells were pelleted by centrifugation, washed once, resuspended in PBS and added to the J774A cultures at 4×10⁸ cells/flask (a 10-fold excess assuming that the J774A cells doubled once after plating). The coculture was incubated for 2.5-3.0 hours at 37° C. in normal air. Yeast cells not associated with the adherent macrophages were removed by washing 3× with ice-cold PBS. The macrophages and associated yeast were removed by scraping, and pooled by centrifugation for 1 min. at 500×g. Cell number and viability (using methylene blue) were determined by microscopy. The cell mixture was washed 2× with ice-cold water to lyse the mammalian cells. The resulting cell pellet, consisting mostly of yeast cells, was frozen at −80° C.

RNA was made from the pooled cell pellets using hot acidic phenol and the poly(A) fraction was isolated using the Poly(A)ttract kit (Promega). 2 μg poly(A) RNA per sample was labeled in duplicate as described (Wodicka, L., et al., Nat. Biotechnol., 15:1359-1367 (1997)) and hybridized to the Ye6100 oligonucleotide array set (Affymetrix). Array data was extracted and filtered to remove any genes whose expression did not change at least 2-fold (or 100 units) in the experiment.

C. albicans homologs of ICL1 and MLS1 were identified by searching currently available C. albicans genome sequence data from the Stanford DNA Sequencing and Technology Center (http://sequence-www.stanford.edu/group/candida/index.html). There are single homologs for each gene in currently available data (Genbank accession numbers: CaICL1, AF222905; CaMLS1, AAF34695). For Northern analysis, macrophage interactions were performed as described above using 10⁶ J774A cells in 5 ml media with 2×10⁷ S. cerevisiae (EM93) or C. albicans (SC5314) cells. Control populations were grown for three hours in rich media (YPD), or in tissue culture media without (RPMI) or with (serum) 10% FBS. Species-specific probes were PCR amplified and random primer labelled.

Mutant Construction and Analysis

S. cerevisiae Δicl1 mutants were constructed in the EM93 background using a PCR mediated protocol with a G418-resistance cassette (Wach, A., et al., Yeast, 10:1793-1808 (1994)). Mutants were constructed in both mating types, and mated to produce a homozygous Δicl1/Δicl1 knockout strain (MLY283aα). For C. albicans, an Δicl1 disruption construct was created by inserting a hisG-URA3-hisG cassette (Fonzi, W. A. & Irwin, M. Y., Genetics, 134:717-728 (1993)) at a BglII site in the ICL1 ORF. This construct was linearized, transformed into CAI4 (a Ura-derivative of strain SC5314; Fonzi, W. A. & Irwin, M. Y., Genetics, 134:717-728 (1993); Gillum, A. M., et al., Mol. Gen. Genet., 198:179-182 (1984)), and selected by uracil prototrophy. Accurate integrants were identified by PCR and passaged on 5-FOA medium. A second round of transformation was used to generate two independent homozygous Δicl1/Δicl1 strain (MLC7 and MLC8; MLC7 was used for most experiments reported here). The wild-type ICL1 gene was reintroduced on linearized plasmid pRC2312 (Cannon, R. D., et al., Mol. Gen. Genet., 235:453-457 (1992)) by transformation to produce a complemented strain (MLC10). C. albicans transformations were done as described (Braun, B. R. & Johnson, A. D., Science, 277:105-109 (1997)). Standard media were used (Sherman, F., Methods EnzymoL.,194:3-21 (1991)) and strains were grown at 37° C. unless otherwise indicated.

Murine Virulence Assay

Overnight cultures of C. albicans strains were diluted into fresh YPD and grown for 3-4 hours at 37° C. Cultures were collected by centrifugation and washed with PBS. 5×10⁶ cells were injected into the tail vein of 18-20 week old female BALB/c mice. 10 mice were used per strain. Mice were monitored for three weeks post-injection and moribund animals were euthenized. Animals were cared for according to NIH guidelines.

Results and Discussion

Systematic studies of host-pathogen interactions have been hampered by the lack of genetic tools in C. albicans. For this reason the related but non-pathogenic yeast S. cerevisiae is often used to uncover relevant genes. In vitro, cultured mammalian macrophages readily ingest both S. cerevisiae and C. albicans cells. A population of S. cerevisiae highly enriched for phagocytosed cells was isolated and subjected to whole genome microarray analysis using oligonucleotide-based arrays (Affymetrix). Three hours after initiating the coculture, most of the phagocytosed cells are alive (averaging 67% alive as assayed by methylene blue staining); transcriptional profiling of these cells reveals the response of fungal cells to phagocytosis.

Eleven of the 15 most highly induced S. cerevisiae genes after phagocytosis (Table 1) encode proteins related to the glyoxylate cycle (GC), through which two-carbon compounds are assimilated into the tricarboxylic acid (TCA) cycle. Three of the five GC enzymes are on this list (isocitrate lyase, ICL1; malate synthase, MLS1; malate dehydrogenase, MDH2), and a fourth, citrate synthase (CIT2), is also strongly induced (4.9-fold, ranking 24^{th}). Further, several genes functionally related to the GC are induced, including acetyl-CoA synthase (ACS1); YDR384c, a homolog of the Yarrowia lipolytica Glyoxylate Pathway Regulator (GPR1 (Kujau, M., et al., Yeast, 8:193-203 (1992); Tzschoppe, K., et al., Yeast, 15:1645-1656 (1999)); several transporters and acetyltransferases, used to traffic intermediates of the GC and fatty acid degradation between organelles (CRC1, ACR1, YAT1, and YER024w); and fructose-1,6-bisphosphatase (FBP1). FBP1 is a key regulatory point in gluconeogenesis (Sedivy, J. M. & Fraenkel, D. G., J. Mol. Biol., 186:307-319 (1985)); the production of glucose is the principal function of the GC. Induction of the GC indicates that nutrient acquisition and utilization is the primary focus of yeast cells upon phagocytosis presumably because the phagolysosome is poor in complex carbon compounds.

Although the GC and TCA share common reactions, it is only the isozymes specialized for the GC that are induced (Table 2). The cytosolic isozyme of malate dehydrogenase (MDH2), which preferentially functions in the GC (Minard, K. I. & McAlister-Henn, L., Mol. Cell. Biol., 11:370-380 (1991)), is induced 15.6-fold. By contrast, the mitochondrial (MDH1) and peroxisomal (MDH3) isozymes are not induced. Of the three citrate synthase isoforms, only the GC-specific one (CIT2) is induced. In control array experiments, expression of GC enzymes were not changed significantly in response to conditioned media, oxidative stress or contact with heat killed macrophages. Thus, phagocytosis specifically upregulates the GC and its accessory proteins. It should be noted that this metabolic response takes precedence over any conventional stress response which indicates that nutrient deprivation is the primary “stress” that confronts these cells.

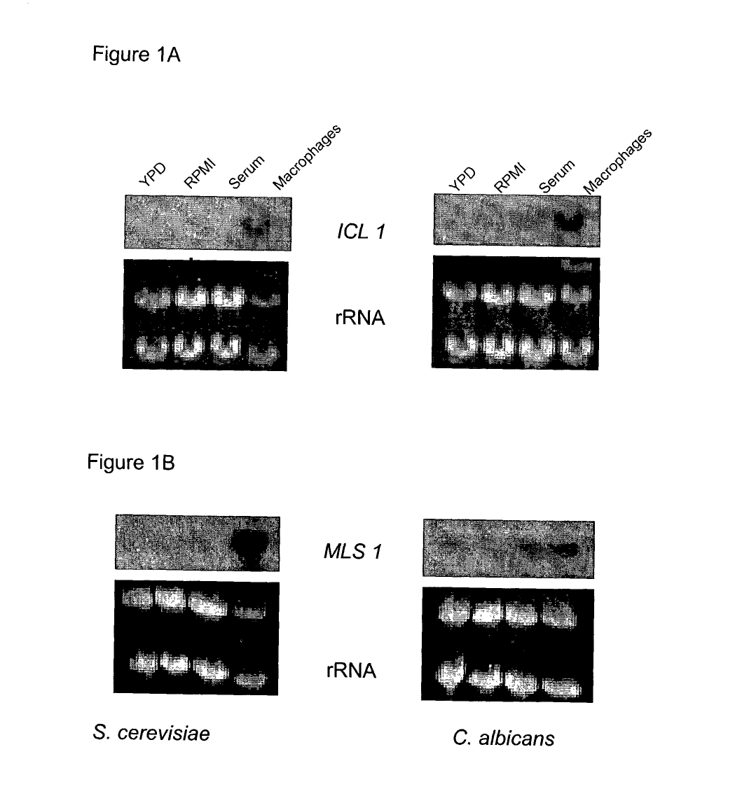

As described herein, the C. albicans genes for isocitrate lyase (CaICL1) and malate synthase (CaMLS1), the only enzymes whose activity is both specific and limited to the glyoxylate cycle, were cloned. Both genes share significant homology with proteins from fungi, plants, and bacteria, but importantly, not mammals which do not have the GC. Northern analysis of RNA from both S. cerevisiae and C. albicans cells grown in the presence of macrophages shows that in both organisms the ICL1 or MLS1 (FIGS. 1A, 1B) genes are significantly induced by macrophage contact when compared to cells grown in media alone. Thus, the induction of the glyoxylate enzymes is a conserved response to phagocytosis in these two yeasts, which diverged from a common ancestor an estimated 150 million years ago.

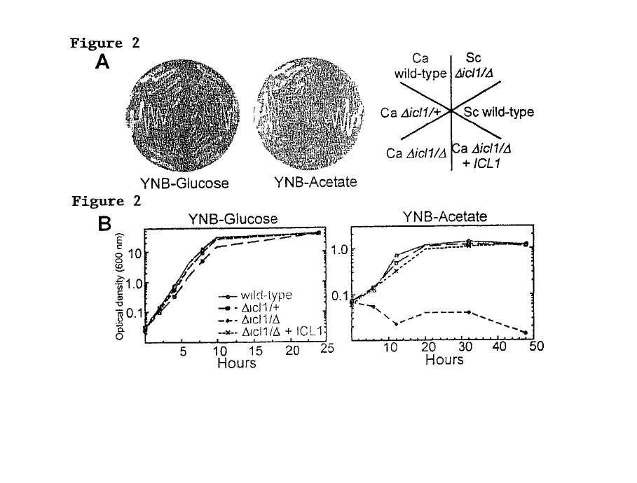

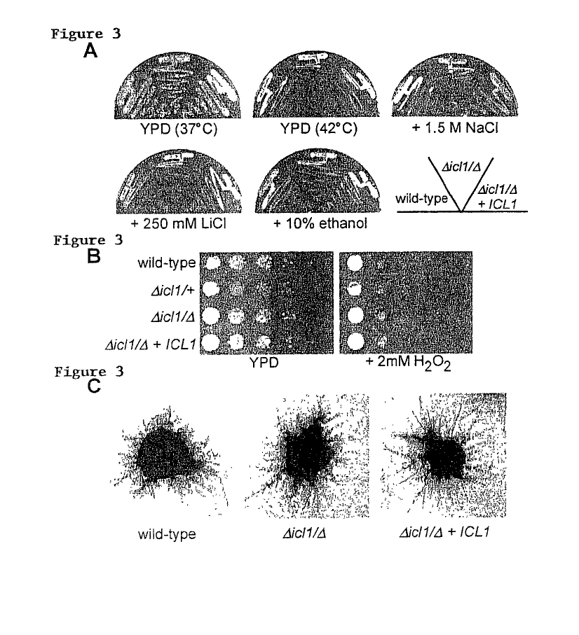

Mutant strains of both S. cerevisiae and C. albicans lacking ICL1 were constructed. In both organisms the ici1 mutant strains fail to utilize acetate or ethanol as carbon sources (FIG. 2A and data not shown). In C. albicans, both the heterozygous strain (icl1/+) and a homozygous mutant in which ICL1 has been reintroduced (Δicl1 Δicl1/+ICL1) grow as well as a wild-type strain on acetate media (FIGS. 2A, 2B). The growth rates of the C. albicans icl1/icl1 strain is not significantly different from the parent strain on rich (YP-Dextrose) media (FIG. 2B), nor is the strain any more sensitive to a variety of in vivo stresses, including salt, heat shock, ethanol (assayed on glucose media), or oxidative stress (FIGS. 3A, 3B). icl1/icl1 strains form filaments (on solid medium) and form germ tubes (in liquid medium) in response to serum or neutral pH (FIG. 3C, data not shown).

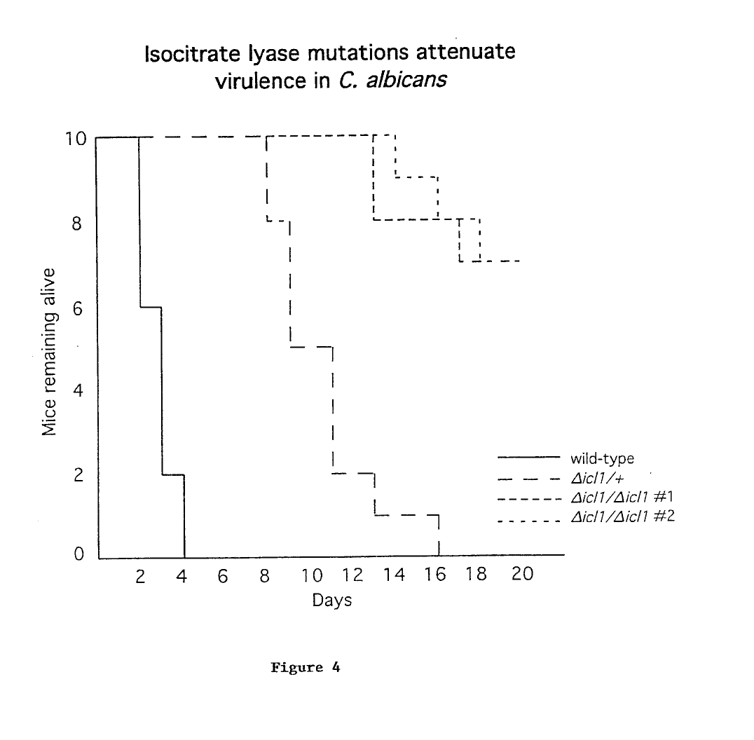

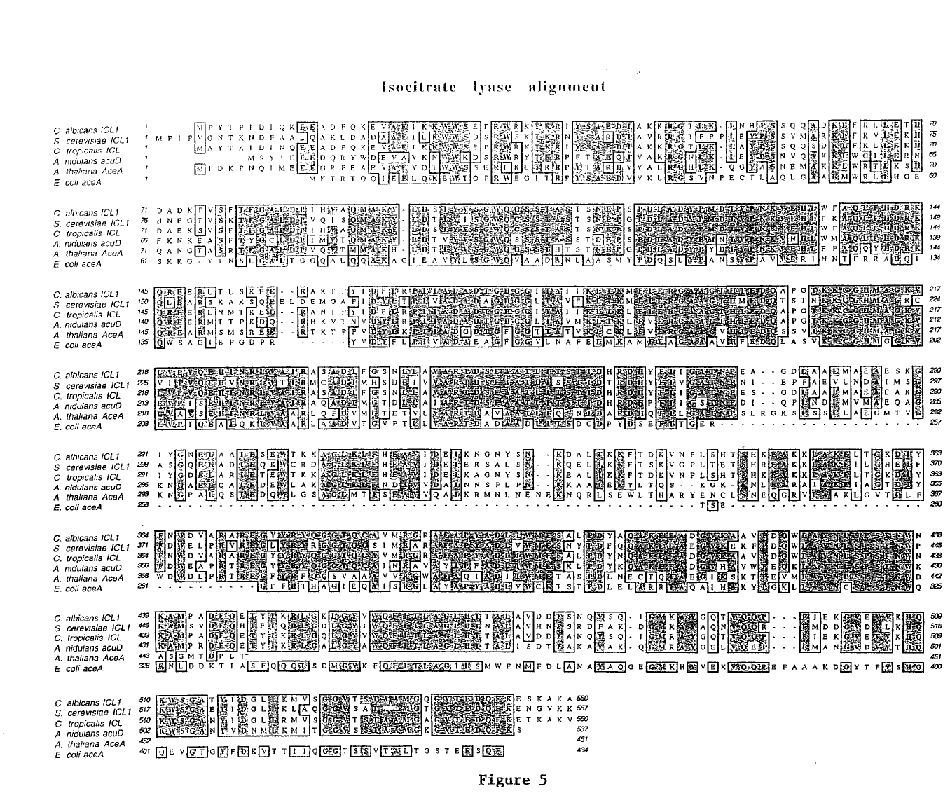

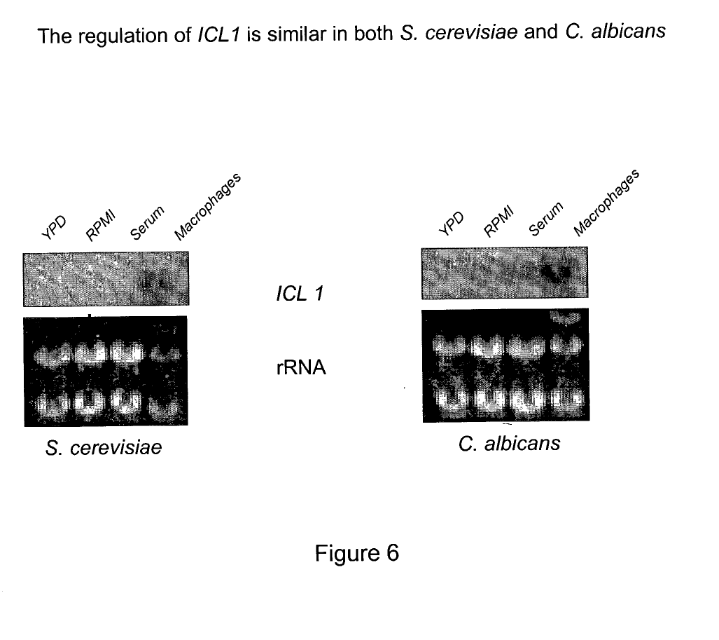

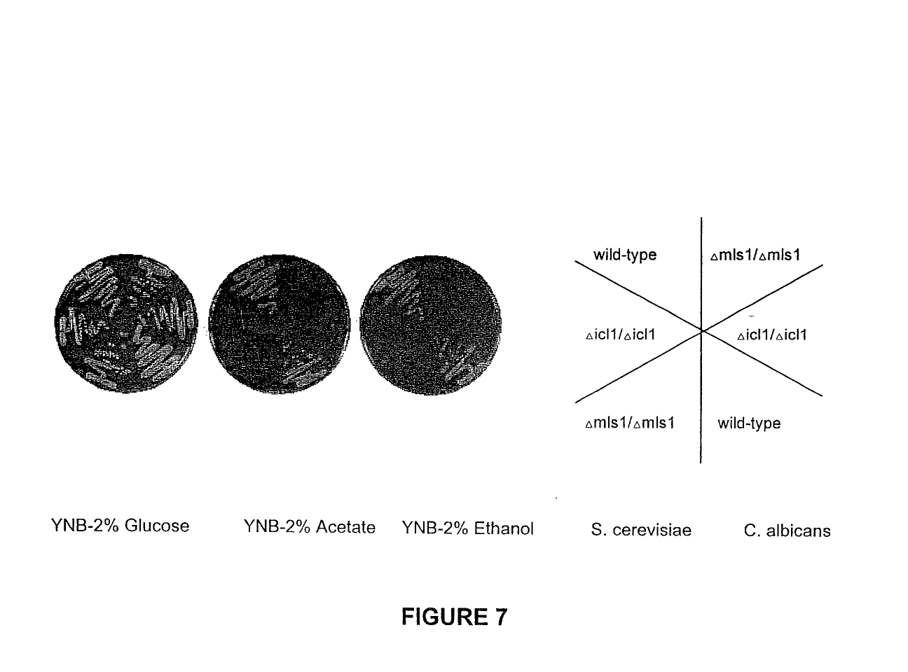

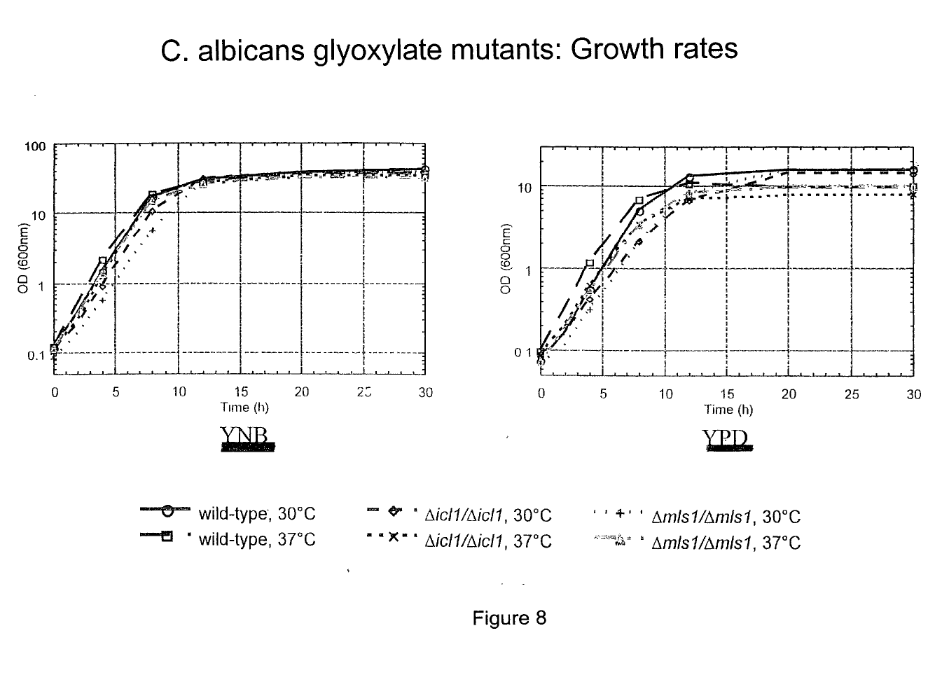

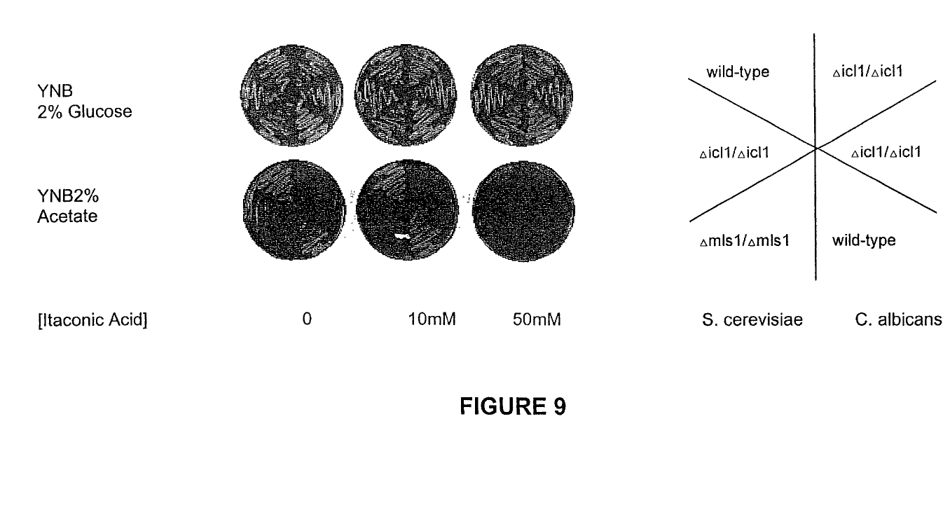

The virulence of these C. albicans strains was tested in a mouse model of systemic candidiasis. Mice injected with wild-type C. albicans strain SC5314 succumb rapidly to the infection (median survival of 3 days; FIG. 4), whereas mice injected with two independently constructed Δicl1/Δicl1 strains survived longer. At day 28, 7/10 of the animals injected with one strain (MLC7) remained alive as did 6/10 of an independent homozygous mutant (MLC8). Infection with the heterozygote (Δicl1/+) resulted in an intermediate mortality (median of 8 days). Thus, isocitrate lyase is not only induced by macrophage phagocytosis, but is also essential for full virulence in this fungal pathogen.

The data from the genome arrays and virulence studies described herein indicate that microbes find the inside of a macrophage to be a glucose-deficient environment. Glucose is required for the synthesis of many macromolecules necessary for proliferation, including ribose and deoxyribose. It is likely that the phagolysosome is a site of fatty acid breakdown, and is thus rich in acetyl-CoA, the endpoint of this process. Acetyl-CoA can only be assimilated via the GC, which bypasses the catabolic steps of the TCA cycle, thus the GC is the only route to the synthesis of glucose in this environment. Although the glyoxylate pathway is necessary for virulence, it is clearly not sufficient. Both Saccharomyces and Candida induce the GC upon macrophage contact, yet only Candida virulent.

Genes encoding the GC have now been shown to be required for virulence in both a bacterium (M. tuberculosis), and a fungus (C. albicans) that can survive inside a macrophage. Inhibitors of the GC pathway should block nutrient availability and prevent survival of these pathogens inside the macrophage. Compounds that inhibit nutrient availability have been developed into effective herbicides (glyphosate, imidizolinones, etc.) because their targets are enzymes produced by plants but not by animals. As the enzymes of the GC are also not found in mammals, they are prime targets for antibacterial and antifungal agents.

TABLE 1
Genes induced by phagocytosis in S. cerevisiae

| Gene | ORF | YPD | Serum | Macrophage | Description |
|---|---|---|---|---|---|
| YAT1 | YAR035W | 0.9 | 1.0 | 36.6 | Outer carnithine acetyltransferase, |
| | | | | | mitochondrial |
| ORF | YMR031C | 5.1 | 1.0 | 36.1 | Unknown |
| ICL1 | YER065C | 0.2 | 1.0 | 22.7 | Isocitrate lyase, peroxisomal (glyoxylate |
| | | | | | cycle) |
| MLS1 | YNL117W | 0.2 | 1.0 | 22.5 | Malate Synthase (glyoxylate cycle) |
| MDH2 | YOL126C | 0.3 | 1.0 | 15.7 | Malate dehydrogenase, cystolic |
| | | | | | (glyoxylate cycle) |
| NCE3 | YNL036W | 1.5 | 1.0 | 13.8 | Similar to carbonic anhydrase; substrate |
| | | | | | for non-classical export pathway |
| ORF | YDR384C | 0.4 | 1.0 | 12.1 | Similar to Y. lipolytica Gpr1p |
| ORF | YKL187C | 2.2 | 1.0 | 11.6 | Similar to 4-mycarosyl isovaleryl-CoA |
| | | | | | transferase; induced by glycerol, oleate |
| FBP1 | YLR377C | 0.1 | 1.0 | 10.8 | Fructose-1,6-bisphosphatase |
| ORF | YMR118C | 0.2 | 1.0 | 10.3 | Succinate dehydrogenase; similar to |
| | | | | | Sdh3p |
| ORF | YER024W | 0.3 | 1.0 | 10.2 | Similar to Yat1p |
| SPS100 | YHR139C | 0.8 | 1.0 | 9.8 | Sporulation specific protein; induced by |
| | | | | | ethanol |
| ACS1 | YAL054C | 0.2 | 1.0 | 8.7 | Acetyl-CoA synthetase |
| CRC1 | YOR100C | 0.1 | 1.0 | 8.1 | Mitochondrial Carrier Family (MCF); |
| | | | | | involved in carnithine transport |
| ACR1 | YJR095W | 0.0 | 1.0 | 6.8 | Mitochondrial succinate-fumarate |
| | | | | | transporter (MCF family) |

Values are fold-induction compared to expression in tissue culture medium plus serum (Serum) for cells grown in rich medium (YPD) or the phagocytosed cell population (Macrophage). Values represent the average of duplicate array experiments. The Description column is derived from the Yeast Protein Database maintained by Proteome, Inc. (www.proteome.com/YPDhome.html).

TABLE 2
Expression of Glyoxylate and TCA enzymes upon phagocytosis

| | | | |
|---|---|---|---|
| Gene | ORF | Induction | Description |

Glyoxylate Specific Enzymes

| | | | |
|---|---|---|---|
| MLS1 | YNL117w | 22.7 | Malate synthase |
| ICL1 | YER065c | 22.4 | Isocitrate lyase |
| MDH2 | YOL162c | 15.6 | Malate dehydrogenase, cytosolic |
| CIT2 | YCR005c | 4.9 | Citrate synthase, peroxisomal |

TCA Specific Enzymes

| | | | |
|---|---|---|---|
| SDH1 | YKL148c | 1.9 | Succinate dehydrogenase, flavoprotein |
| SDH4 | YDR178w | 1.7 | Succinate dehydrogenase, membrane |
| KGD1 | YIL125w | 1.4 | α-ketoglutarate dehydrogenase, E1 |
| CIT1 | YNR001c | 1.1 | Citrate synthase, mitochondrial |
| SDH3 | YKL141w | 1.0 | Succinate dehydrogenase, membrane |
| SDH2 | YLL041c | 0.9 | Succinate dehydrogenase, iron-sulfur |
| ACO1* | YLR304c | 0.8 | Aconitase |
| MDH3 | YDL087c | 0.8 | Malate dehydrogenase, peroxisomal |
| MDH1 | YKL085c | 0.7 | Malate dehydrogenase, mitochondrial |
| FUM1 | YPL262w | 0.6 | Fumarate hydratase |
| IDH1 | YNL037c | 0.4 | Isocitrate dehydrogenase, subunit 1 |

*Aconitase (ACO1) is used in both the TCA cycle and the glyoxylate cycle. TCA enzymes CIT3 (citrate synthase), LSC1, LSC2 (succinyl-CoA synthase), IDH2 (isocitrate dehydrogenase, subunit 2) and KGD2 (α-ketoglutarate dehydrogenase) did not meet the filter requirements set (see Methods).

TABLE 3
S. Cerevisiae genes induced by phagocytosis

| Rank | Gene | ORF | YPD | Serum | Internal | YPD |
|---|---|---|---|---|---|---|
| 1 | YAT1 | YAR035W | 0.9 | 1.0 | 36.6 | Outer carnitine |
| | | | | | | acetyltransferase, |
| | | | | | | mitochondrial |
| 2 | ORF | YMR031C | 5.1 | 1.0 | 36.1 | Protein of unknown |
| | | | | | | function |
| 3 | ICL1 | YER065C | 0.2 | 1.0 | 22.7 | Isocitrate lyase |
| | | | | | | peroxisomal (glyoxylate |
| | | | | | | cycle) |
| 4 | MLS1 | YNL117W | 0.2 | 1.0 | 22.5 | Malate synthase 1 |
| | | | | | | (glyoxylate cycle) |
| 5 | MDH2 | YOL126C | 0.3 | 1.0 | 15.7 | Malate dehydrogenase |
| | | | | | | cytosolic (glyoxylate |
| | | | | | | cycle) |
| 6 | NCE3 | YNL036W | 1.5 | 1.0 | 13.8 | Involved in a non- |
| | | | | | | classical protein export |
| | | | | | | pathway |
| 7 | ORF | YDR384C | 0.4 | 1.0 | 12.1 | Protein with similarity to |
| | | | | | | Y. lipolytica Gpr1p |
| 8 | ORF | YKL187C | 2.2 | 1.0 | 11.6 | Similar to 4-mycarosyl |
| | | | | | | isovaleryl-CoA |
| | | | | | | transferase |
| 9 | FBP1 | YLR377C | 0.1 | 1.0 | 10.8 | Fructose-1,6- |
| | | | | | | biphosphatase; |
| | | | | | | gluconeogenic enzyme |
| 10 | ORF | YMR118C | 0.2 | 1.0 | 10.3 | Succinate dehydrogenase |
| | | | | | | protein, similarity to |
| | | | | | | Sdh3p |
| 11 | ORF | YER024W | 0.3 | 1.0 | 10.2 | Protein with similarity to |
| | | | | | | Yat1p |
| 12 | SPS100 | YHR139C | 0.8 | 1.0 | 9.8 | Sporulation specific |
| | | | | | | protein; spore wall |
| | | | | | | formation |
| 13 | ACS1 | YAL054C | 0.2 | 1.0 | 8.7 | Acetyl-CoA synthetase |
| 14 | ORF | YOR100C | 0.1 | 1.0 | 8.1 | Similar to the |
| | | | | | | mitochondrial carrier |
| | | | | | | (MCF) family |
| 15 | ACR1 | YJR095W | 0.0 | 1.0 | 6.8 | Mitochondrial membrane |
| | | | | | | succinate-fumarate |
| | | | | | | transporter |
| 16 | ORF | YMR303C | 0.1 | 1.0 | 6.3 | Alcohol dehydrogenase |
| | | | | | | II |
| 17 | FOX2 | YKR009C | 0.1 | 1.0 | 6.2 | 3-hydroxyacyl-CoA |
| | | | | | | epimerase |
| 18 | ORF | YCR010C | 0.0 | 1.0 | 5.6 | Protein of unknown |
| | | | | | | function |
| 19 | ORF | YMR034C | 0.4 | 1.0 | 5.6 | Protein of unknown |
| | | | | | | function |
| 20 | ORF | YNL014W | 0.2 | 1.0 | 5.3 | Translation elongation |
| | | | | | | factor EF-3B |
| 21 | ORF | YLR164W | 0.2 | 1.0 | 5.3 | Protein with strong |
| | | | | | | similarity to Sdh4p |
| 22 | CIT2 | YCR005C | 0.2 | 1.0 | 4.9 | Citrate synthase, |
| | | | | | | peroxisomal (Glyoxylate |
| | | | | | | cycle?) |
| 23 | SSA3 | YBL075C | 0.1 | 1.0 | 4.5 | Chaperone of the HSP70 |
| | | | | | | family |
| 24 | ORF | YPR006C | 0.2 | 1.0 | 4.4 | Isocitrate lyase, may be |
| | | | | | | nonfunctional |
| 25 | HSP104 | YLL026W | 1.9 | 1.0 | 4.4 | Heat shock protein |

TABLE 4
Induction by phagocytosis is specific

| | |
|---|---|
| Glyoxylate components | TCA cycle components |

| Gene | Induction | Gene | Induction |
|---|---|---|---|
| ILC1 | 22.7 | SDH1 | 1.9 |
| MLS1 | 22.4 | SDH4 | 1.7 |
| MDH2 | 15.6 | KGD1 | 1.4 |
| CIT2 | 4.9 | CIT | 1.1 |
| | | SDH3 | 1.0 |
| | | SDH2 | 0.9 |
| | | ACO1 | 0.8 |
| | | MDH3 | 0.8 |
| | | MDH1 | 0.7 |
| | | FUM1 | 0.6 |
| | | IDH1 | 0.4 |

TABLE 5
CalCL1 and CaMLS1 are similar to other glyoxylate enzymes

| Isocitrate lyase | | | Malate synthase | | |
|---|---|---|---|---|---|
| Organism | Protein | % Identity | Organism | Protein | % Identity |
| C. tropicalis | ICL1 | 94.5 | C. tropicalis | PMS2 | 93.6 |
| A. nidulans | AcuD | 66.9 | S. cerevisiae | MLS1 | 51.5 |
| S. cerevisiae | ICL1 | 65.7 | Z. mays | MLS1 | 41.2 |
| E. coli | AceA | 36.6 | E. coli | MasZ | 12.9 |

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of identifying a drug that inhibits an enzyme involved in the glyoxylate cycle of a microorganism, comprising:
a) contacting the microorganism with a drug to be assessed for its ability to inhibit an enzyme involved in the glyoxylate pathway of the microorganism;
b) determining the ability of the microorganism to utilize two-carbon compounds as the sole carbon source, wherein if the ability of the microorganism to utilize two-carbon compounds is reduced in the presence of the drug to be assessed, then the drug inhibits an enzyme involved in the glyoxylate cycle of the microorganism.

2. The method of claim 1 wherein the microorganism is M. tuberculosis.

3. The method of claim 1 wherein the microorganism is a fungus.

4. The method of claim 3 wherein the fungus is a filamentous fungus.

5. The method of claim 4 wherein the filamentous fungus is a member of a species selected from the group consisting of: Acremonium species, Aspergillus species, Claviceps species, Collertortichum species, Fusarium species, Monascue species, Neurospora species, Nodulisporium species, Penicillium species, Pestalotiopsis species, Taxomyces species, Tolypocladium species and Trichoderma species.

6. The method of claim 4 wherein the fungus is yeast.

7. The method of claim 6 wherein the yeast is selected from the group consisting of: S. cerevisiae and C. albicans.

8. A method of identifying a drug that inhibits the virulence of a fungus comprising:
a) contacting the fungus with a drug to be assessed;
b) determining whether an enzyme involved in the glyoxylate pathway of the fungus is inhibited in the presence of the drug wherein if an enzyme of the glyoxylate pathway of the fungus is inhibited in the presence of the drug, then the drug inhibits virulence of the fungus.

9. The method of claim 8 wherein the fungus is a filamentous fungus.

10. The method of claim 9 wherein the filamentous fungus is a member of a species selected from the group consisting of: Acremonium species, Aspergillus species, Claviceps species, Collertortichum species, Fusarium species, Monascue species, Neurospora species, Nodulisporium species, Penicillium species, Pestalotiopsis species, Taxomyces species, Tolypocladium species and Trichoderma species.

11. The method of claim 8 wherein the fungus is yeast.

12. The method of claim 11 wherein the yeast is selected from the group consisting of: S. cerevisiae and C. albicans.

13. The method of claim 12 wherein the enzyme of the glyoxylate pathway is selected from the group consisting of: isocitrate lyase (ICL1), malate synthase synthase (MLS1), malate dehydrogenase (MDH2), citrate synthase (CIT2), acetyl-CoA synthase (ACS1), CRC1, ACR1, YAT1, YER024w, YDR384c and fructose-1,6-biphosphatase (FBP1).

14. The method of claim 8 wherein whether an enzyme of the glyoxylate pathway of the fungus is inhibited in the presence of the drug is determined using an infectivity assay.

15. A method of identifying a drug that inhibits the virulence of a yeast comprising:
a) contacting the yeast with a drug to be assessed;
b) determining whether an enzyme involved in the glyoxylate pathway of the yeast is inhibited in the presence of the drug wherein if an enzyme of the glyoxylate pathway of the yeast is inhibited in the presence of the drug, then the drug inhibits virulence of the yeast.

16. The method of claim 15 wherein the yeast is selected from the group consisting of: S. cerevisiae and C. albicans.

17. The method of claim 16 wherein the enzyme of the glyoxylate pathway is selected from the group consisting of: isocitrate lyase (ICL1), malate synthase synthase (MLS1), malate dehydrogenase (MDH2), citrate synthase (CIT2), acetyl-CoA synthase (ACS1), CRC1, ACR1, YAT1, YER024w, YDR384c and fructose-1,6-biphosphatase (FBP1).

18. The method of claim 15 wherein whether an enzyme of the glyoxylate pathway of the yeast is inhibited in the presence of the drug is determined using an infectivity assay.

19. A method of identifying a drug that inhibits the virulence of C. albicans comprising:
a) contacting the C. albicans with a drug to be assessed;
b) determining whether an enzyme involved in the glyoxylate pathway of the C. albicans is inhibited in the presence of the drug wherein if an enzyme of the glyoxylate pathway of the C. albicans is inhibited in the presence of the drug, then the drug inhibits virulence of the C. albicans.

20. The method of claim 19 wherein the enzyme of the glyoxylate pathway is selected from the group consisting of: isocitrate lyase (ICL1), malate synthase synthase (MLS1), malate dehydrogenase (MDH2), citrate synthase (CIT2), acetyl-CoA synthase (ACS1), CRC1, ACR1, YAT1, YER024w, YDR384c and fructose-1,6-biphosphatase (FBP1).

21. The method of claim 19 wherein whether an enzyme of the glyoxylate pathway of the C. albicans is inhibited in the presence of the drug is determined using an infectivity assay.

22. A method of identifying a drug that inhibits the virulence of C. albicans comprising:
a) contacting the C. albicans with a drug to be assessed;
b) determining whether isocitrate lyase of the C. albicans is inhibited in the presence of the drug wherein if the isocitrate lyase of the C. albicans is inhibited in the presence of the drug, then the drug inhibits virulence of the C. albicans.

23. The method of claim 22 wherein whether the isocitrate lyase of the glyoxylate pathway of the C. albicans is inhibited in the presence of the drug is determined using an infectivity assay.

24. A method of identifying a drug that inhibits the virulence of C. albicans comprising:
a) contacting the C. albicans with a drug to be assessed;
b) determining whether malate synthase of the C. albicans is inhibited in the presence of the drug wherein if the malate synthase of the C. albicans is inhibited in the presence of the drug, then the drug inhibits virulence of the C. albicans.

25. The method of claim 24 wherein whether an enzyme of the glyoxylate pathway of the C. albicans is inhibited in the presence of the drug is determined using an infectivity assay.

26. A method of treating an individual who is susceptible to fungal infection comprising administering to the individual a drug identified by the method of claim 8.

27. A method of treating an individual who is susceptible to a yeast infection comprising administering to the individual a drug identified by the method of claim 15.

28. A method of treating an individual who is susceptible to a C. albicans infection comprising administering to the individual a drug identified by the method of claim 19.

29. A method of treating an individual who is susceptible to a C. albicans infection comprising administering to the individual a drug identified by the method of claim 22.

30. A method of treating an individual who is susceptible to a C. albicans infection comprising administering to the individual a drug identified by the method of claim 24.

31. The method of claim 26 wherein the individual is undergoing immunosuppressive or chemotherapeutic treatment.

32. The method of claim 26 wherein the individual is neutropenic.

33. The method of claim 32 wherein the individual is undergoing immunosuppressive or chemotherapeutic treatment.

34. The method of claim 32 wherein the individual has a disease selected from the group consisting of leukemia and lymphoma.

35. The method of claim 26 the individual is T-cell deficient.

36. The method of claim 35 wherein the individual is infected with HIV.

37. A method of treating a fungus infection in an individual comprising administering to the individual a therapeutically effective amount of a drug that inhibits an enzyme involved in the glyoxylate cycle of the fungus.

38. The method of claim 37 wherein the fungus is a filamentous fungus.

39. The method of claim 38 wherein the filamentous fungus is a member of a species selected from the group consisting of: Acremonium species, Aspergillus species, Claviceps species, Collertortichum species, Fusarium species, Monascue species, Neurospora species, Nodulisporium species, Penicillium species, Pestalotiopsis species, Taxomyces species, Tolypocladium species and Trichoderma species.

40. The method of claim 37 wherein the fungus is yeast.

41. The method of claim 40 wherein the yeast is selected from the group consisting of S. cerevisiae and C. albicans.

42. The method of claim 37 wherein the enzyme of the glyoxylate pathway is selected from the group consisting of: isocitrate lyase (ICL1), malate synthase synthase (MLS1), malate dehydrogenase (MDH2), citrate synthase (CIT2), acetyl-CoA synthase (ACS1), CRC1, ACR1, YAT1, YER024w, YDR384c and fructose-1,6-biphosphatase (FBP1).
